(19) United States
(12) Patent Application Publication
(10) Pub. No.: US 2020/0155589 A1
(43) Pub. Date: May 21, 2020

(54) Composition For Enchanced Recovery After Surgery (ERAS)

(71) Applicant: Gerald Haase, Denver, CO (US)

(72) Inventor: Gerald Haase, Denver, CO (US)

(21) Appl. No.: 16/501,845

(22) Filed: Jun. 17, 2019

Related U.S. Application Data
(60) Provisional application No. 62/685,498, filed on Jun. 15, 2018.

(51) Int. Cl. A61K 31/7088; A61K 31/522; A61K 45/06; A61P 39/06

(52) U.S. Cl. CPC A61K 31/7088; A61P 39/06; A61K 45/06; A61K 31/522

(57) ABSTRACT

A formulation comprising at least one phytonutrient, at least one mineral, at least one dietary vitamin, at least one antioxidant, at least one carbohydrate, at least one electrolyte, at least one amino acid, and at least one other nutrient.

### RELATED APPLICATION

This is a utility of the provisional application of U.S. application Ser. No. 62/685,498 entitled “COMPOSITIONS OF NUTRIENTS AND PHYTOCHEMICALS FOR PREOPERATIVE FUNCTIONAL BEVERAGES IN ENHANCED RECOVERY AFTER SURGERY (ERAS) PROTOCOLS”.

### FIELD OF THE INVENTION

This invention relates to methods and formulations including macronutrients (carbohydrates), phytochemicals from plant sources, and other micronutrients including electrolytes, minerals, dietary vitamins, antioxidants, amino acids, nucleotides and other miscellaneous nutrients adapted for preoperative functional beverages to be utilized in Enhanced Recovery After Surgery (ERAS) clinical protocols.

### BACKGROUND OF THE INVENTION

Surgical procedures have always represented major therapeutic interventions in clinical medicine. The utility of operative treatment has continued to grow worldwide as technical improvements have increased its application and efficacy while decreasing related complications. However, relative to the more than 320 million operations performed annually, about 25% are still associated with patient complications. In concert with this explosive utilization of surgery, it is mandatory that continued refinements in perioperative care improve overall patient outcomes. One of the more dramatic evolutions in recent patient management has been the emerging concept of Enhanced Recovery After Surgery, (acronym ERAS), protocols. This approach originated as a collaboration in several European surgical centers with the intent of standardizing “best practices” from the known surgical literature. The impetus for this effort resulted from groundbreaking research demonstrating that safe early discharge of patients undergoing complex bowel surgery was possible. As standard protocol guidelines became more widely accepted, multicenter experience documented lower complication rates and improved long-term outcomes. Postoperative length of stay was significantly decreased. Eventually, it also became clear that significant cost savings could be achieved by implementation of ERAS programs in single institutions as well as larger health care systems. These findings in public and private integrated health care models extended to favorable outcomes in terms of complications, opioid use and readmissions. Presently, according to a 2017 presentation from the ERAS USA Society, there are over 45,000 patient records available in the international Quality Registry.

A major component of ERAS protocols addressed the issue of optimal patient preparation for a surgical procedure. It is well known that anesthesia and surgery are generally poorly understood by patients and are a source of anxiety. These feelings of fear are exacerbated by the preoperative starvation of patients as had been the traditional practice. Experimental data from animal studies demonstrate that starvation is metabolically detrimental. In fact, a substantial percentage of patients were already nutritionally deficient prior to surgery and their hydration status could be suboptimal. Even mild dehydration impairs mood and neurologic function. In addition, thirst and sleep loss related to these factors causes anxiety and cognitive dysfunction. Allowing clear liquid intake in the form of carbohydrate loading would address these issues and provide other health benefits. In fact, less postoperative nausea and vomiting are experienced by patients who are not fasted prior to surgery. The proven safety of oral feeding soon after surgery may have contributed to this adoption of permissive guidelines mandating preoperative consumption of liquids.

In addition to decreasing the surgical stress related to hunger, thirst and anxiety, preoperative carbohydrate loading provided important metabolic advantages. It is known that postoperative insulin resistance occurs in response to fasting and surgical stress and renders management of proper glucose levels difficult. Postoperative hyperglycemia and its severity parallels hospital length of stay and further complications. Carbohydrate loading reduces this insulin insensitivity and decreases the risks associated with hyperglycemia while not altering gastric emptying. Preoperative oral clear liquid intake improves patient functional status and recovery involving several factors. Chronic inflammation also contributes to length of hospitalization and patient morbidity. Conversely, adequate nutrition and preoperative carbohydrate loading reduce hospital stay, infectious complications and patient distress from hunger and anxiety (39-41). With reduced anxiety alone, functional status is improved.

Of further benefit, the insulin sensitivity effect of preoperative liquid carbohydrate loading may reduce the need for traditional “tight glucose level control” by exogenous insulin administration and the resulting risks of hypoglycemia. The safety of this approach has also been demonstrated in diabetics as these patients with insulin resistance are not adversely affected by the oral carbohydrate load. Insulin resistance may also correlate with protein catabolism and decrease in muscle mass. In fact, preoperative oral glucose improves preservation of muscle mass after surgery. This has relevance in that perioperative improvement in endurance and functional capacity have positive influence on patient outcomes. Of critical importance, there were no adverse effects on gastric pH or volume and no increase in adverse events such as pulmonary aspiration reported after preoperative liquid intake. In addition, minimally invasive surgical techniques have supplanted many “open” procedures for therapeutic operative interventions. It is encouraging to recognize that ERAS protocols and preoperative oral carbohydrate loading are also applicable to laparoscopic surgery.

In summary, it has now been established that ERAS protocols can be effectively implemented in a variety of health care systems and significant positive economic impact can derived in both direct and indirect costs of care. It is in this favorable financial environment that this invention is particularly relevant and clinically unique with even greater efficacy potential than is already evident. In addition, the approach is widely applicable to use in most surgical specialties and operative situations. Of particular interest, and along with the expected shorter length of stay and lower costs, are multiple studies demonstrating improved time to return of intestinal function, improved inpatient quality of life scores, lower complication rates and fewer hospital re-admissions. The overall ERAS strategy and components of this invention as the preoperative carbohydrate loading beverage have remarkable potential effects on perioperative metabolism. Surgery-related catabolism is decreased preserving muscle mass and several markers of inflammatory responses are reduced. Also, the ingredients may have immune-enhancing effects and improve wound healing as well as reducing pulmonary pressures after surgery.

This invention seeks to exploit nutritional and antioxidant science to provide a unique, effective clear liquid preoperative beverage. Oxygen utilization and related normal human metabolism generate free radicals that are potentially toxic by causing a condition known as oxidative stress. This oxidative damage may injure cells, tissues, organs and body proteins, lipids and DNA. Oxidative stress obviously also occurs during surgical procedures and several detrimental effects have already been described. While antioxidants are known protective agents, other cellular processes must also be considered as targets for therapeutic intervention since there is increasing recent recognition of the potential for natural substances to be beneficial. In addition, multiple inorganic and organic free radicals are derived from oxygen and nitrogen including anions, organic free radicals, nitric oxide and peroxynitrite. Therefore, while injury may occur from many sources, the pathways of damage involve a few common mechanisms. This invention utilizes the current information relative to the benefits of the included micronutrients in reducing perioperative physical stressors and neuropsychological factors.

In addition to excess free radicals, acute and chronic inflammatory reactions also contribute to the spectrum of injury. Reactive oxygen and nitrogen species and pro-inflammatory cytokines such as interleukin-6 and tumor necrosis factor-alpha, as well as adhesion molecules, complement proteins and prostaglandin E2 are released during these reactions. These released substances are very injurious to cells and organs. It is known that free radicals, reactive species, pro-inflammatory cytokines and the other molecules may be etiological factors in the initiation and/or progression of acute adverse health effects from surgical interventions. Oxidative damage has also been related to impaired immune function and the aging process.

With this background, the current invention employs the established tenets of functional foods in the prevention or reduction of adverse health outcomes in a variety of clinical scenarios. Specifically, functional beverages are emerging as an efficacious platform to deliver important nutrients and bioactive compounds. It is also the intent of the invention to overcome the limitations of other ERAS supplement preparations. For example, many commercially-available products employ maltodextrin as the carbohydrate source. This polysaccharide is artificially processed from starch (usually corn or rice) and has a high glycemic index that may lead to spikes in blood glucose level. It also may be associated with undesirable bloating and flatulence. In addition, recent research has shown that this carbohydrate may enhance invasive E. coli activity in susceptible individuals and promote dysbiosis.

Conversely, the complex formulations herein aim to maximize the potential beneficial health effects by combining a broad spectrum of micronutrients from multiple sources including carbohydrates, phytochemicals, electrolytes, minerals, vitamins, antioxidants, amino acids, nucleotides and other miscellaneous nutrient agents. It was developed for broad human use from age four years and older for those individuals involved in ERAS protocols as part of their perioperative care. The formulation is intended to improve outcomes by modulating adverse metabolic effects, reducing oxidative damage and inflammation while supporting the body's immune function and enhancing neuroprotection.

### SUMMARY OF THE INVENTION

1. In one embodiment, the present invention relates to a formulation comprising at least one phytonutrient, at least one mineral, at least one dietary vitamin, at least one antioxidant, at least one carbohydrate, at least one electrolyte, at least one amino acid, and at least one other nutrient, and mixtures and combinations thereof. In another embodiment, the present invention relates to a method of manufacturing a formulation, said method comprises admixing at least one phytonutrient, at least one mineral, at least one dietary vitamin, at least one antioxidant, at least one carbohydrate, at least one electrolyte, at least one amino acid, and at least one other nutrient. In still another embodiment, the formulation further comprises caffeine. In yet another embodiment, the formulation further comprises Nucleotides complexes. In a further embodiment, the present invention provides for a dietary supplement of a complex nutritional formulation and combination comprising natural and/or organic cane sugar, brown rice water, inulin, resistant starches, coconut water, blueberry, curcumin, monk fruit, stevia, nitrates (beetroot), sodium, potassium, magnesium, selenium, zinc, vitamin A, vitamin C, vitamin B6, folate, vitamin B12, carotenoids, N-acetyl cysteine, alpha lipoic acid, L-arginine, L-carnitine, L-citrulline, L-glutamine, whey protein, nucleotides (adenosine, guanosine, cytidine, uridine, thymidine) with D-ribose, caffeine and omega-3 fatty acids wherein said formulation is designed as a functional carbohydrate-loading solution for preoperative oral administration as part of an Enhanced Recovery After Surgery (ERAS) clinical therapeutic program. The rationale for the multiple components and the ingredient categories are explained and include:
a) carbohydrates
b) phytonutrients
c) electrolytes
d) mineral co-factors
e) dietary vitamins
f) antioxidants
g) amino acids
h) nucleotides
i) miscellaneous

### DETAILED DESCRIPTION OF THE INVENTION:

As required, detailed embodiments of the present invention are disclosed herein; however, it is to be understood that the disclosed embodiments are merely exemplary of the invention that may be embodied in various forms. The figures are not necessarily to scale, some features may be exaggerated to show details of particular components. Therefore, specific structural and functional details disclosed herein are not to be interpreted as limiting, but merely as a basis for the claims and as a representative basis for teaching one skilled in the art to variously employ the present invention.

The specific example below will enable the invention to be better understood. However, they are given merely by way of guidance and do not imply any limitation.

### Product Formulations by Category and Age

This invention provides novel formulations that are predominantly derived from eight distinct categories as well as miscellaneous ingredients. In some instances, a particular component may exhibit characteristics that would be considered to overlap into more than one category. The combinations listed are further based on age with the primary doses and dose ranges adjusted accordingly. For initial commercial application, the servings are intended to be manufactured in a clear liquid form of appropriate volume.

TABLE 1
STANDARD FORMULATION, ages 14 years and older, comprising of:

| | Primary Dose: | | Range: | |
|---|---|---|---|---|
| Carbohydrates | | | | |
| Cane sugar (organic and/or natural source) | 12 | gms | (10-50 | gms) |
| Coconut water (native) | 360 | gms | (240-1440 | gms) |
| (extract, concentrate) | 18 | gms | (12-72 | gms) |
| D-ribose (see listing below under “Nucleotides” | | | | |
| category) | | | | |
| Resistant starch (see listing below under | | | | |
| “Miscellaneous” category) | | | | |
| Brown rice water (see listing below under | | | | |
| “Miscellaneous” category) | | | | |
| Inulin (see listing below under “Miscellaneous” | | | | |
| category) | | | | |
| Phytonutrients | | | | |
| Blueberry (whole fruit, juice, vinegar, extract, powder- | 800 | mg | (200-1600 | mg) |
| also 4% freeze-dried form, antho-flavonoids) | | | | |
| Curcumin (turmeric root, turmeric extract) | 600 | mg | (100-1800 | mg) |
| Monk fruit (luo han guo, raw whole fruit, extract) | 250 | mg | (10-5,000 | mg) |
| Nitrates (beetroot, powder, extract) | 650 | mg | (100-4,000 | mg) |
| (beetroot juice) | 240 | ml | (120-960 | ml) |
| (sodium nitrate) | 500 | mg | (100-2,500 | mg) |
| Stevia (rebaudioside A, leaf, extract powder) | 100 | mg | (20-2,000 | mg) |
| (liquid drops concentrate) | 30 | mg | (15-100 | mg) |
| Electrolytes | | | | |
| Sodium (chemical or natural form) | 150 | mg | (120-2,000 | mg) |
| Potassium (chemical or natural form) | 750 | mg | (750-5,000 | mg) |
| Mineral Co-Factors | | | | |
| Magnesium (citrate, lactate, gluconate, oxide, carbonate, | 50 | mg | (40-500 | mg) |
| hydroxide, chloride, sulfate, lactic acid, phosphate, | | | | |
| tribasic phosphate, natural forms) | | | | |
| Selenium (L-selenomethionine, sodium selenite/selenate) | 50 | mg | (30-100 | mcg) |
| Zinc (glycinate, gluconate, oxide, sulfate, natural forms) | 3 | mg | (1.1-15 | mg) |
| Dietary Vitamins | | | | |
| Vitamin A (retinyl palmitate, fish oils, natural sources) | 2,500 | IU | (1,250-5,000 | IU) |
| Vitamin C (calcium ascorbate, ascorbic acid, citrus, rose | 250 | mg | (50-2,000 | mg) |
| or berry sources) | | | | |
| Vitamin B6 (pyridoxine hydrochloride or yeast sources) | 5 | mg | (1.0-10 | mg) |
| Vitamin B12 (methylcobalamin, cyanocobalamin, yeast | 5 | mg | (1.0-10 | mg) |
| sources) | | | | |
| Vitamin B9 (folate, folic acid, folacin, yeast or liver | 400 | mg | (100-1,000 | mg) |
| sources) | | | | |
| Antioxidants | | | | |
| N-acetyl cysteine (L-cysteine or natural sources: soy | 125 | mg | (50-500 | mg) |
| extract, sesame or flaxseeds, oat bran, wheat germ, | | | | |
| Swiss cheese, peas or whole grain extracts) | | | | |
| Alpha lipoic acid (R+, R−, yeast or liver sources) | 50 | mg | (25-150 | mg) |
| Carotenoids (natural mixed, beta carotene, sea, algal | 10 | mg | (5-60 | mg) |
| or plant sources) | | | | |
| Amino Acids | | | | |
| Whey protein (amino acid source: native, concentrate, | 6,000 | mg | (2,500-10,000 | mg) |
| isolate, hydrolysate) | | | | |
| L-arginine (chemical or natural form) | 1,500 | mg | (400-6,000 | mg) |
| L-carnitine (fumarate or natural form) | 100 | mg | (50-1,000 | mg) |
| L-citrulline (free or malate) | 3,000 | mg | (500-7,000 | mg) |
| L-glutamine (free, alanyl-L-glutamine, trans alanyl- | 2,500 | mg | (400-10,000 | mg) |
| L-glutamine) | | | | |
| Nucleotides | | | | |
| Complexes (chemical forms or natural sources: e.g. yeast) | 265 | mg | (55-1,500 | mg) |
| (adenosine monophosphate) | 60 | mg | (10-300 | mg) |
| (guanosine monophosphate) | 80 | mg | (16-350 | mg) |
| (cytidine monophosphate) | 50 | mg | (15-300 | mg) |
| (uridine monophosphate) | 50 | mg | (9-300 | mg) |
| (thymidine monophosphate) | 25 | mg | (5-250 | mg) |
| D-ribose (natural source, powder, capsules, tablets) | 3,500 | mg | (2,500-15,000 | mg) |
| Miscellaneous | | | | |
| Caffeine (leaves, nuts, beans, extract) | 125 | mg | (10-750 | mg) |
| Omega-3 fatty acids (oils, seeds, yeast, extract) | 1,000 | mg | (500-6,000 | mg) |
| Resistant starch | 15 | gms | (8-45 | gms) |
| Brown rice water (native) | 360 | gms | (120-1440 | gms) |
| Inulin (chicory, fructans, oligofructose) | 18 | gms | (9-45 | gms) |

For adults utilizing this formulation, it is intended that the commercial product platforms contain at least two constituents from each of at least four ingredient categories.

TABLE 2
YOUTH FORMULATION, ages 4 to 13 years old, comprising of:

| | Primary Dose: | | Range: | |
|---|---|---|---|---|
| Carbohydrates | | | | |
| Cane sugar (organic and/or natural source) | 6 | gms | (1-10 | gms) |
| Coconut water (native) | 120 | gms | (30-240 | gms) |
| (extract, concentrate) | 9 | gms | (2-12 | gms) |
| D-ribose (see listing below under “Nucleotides” | | | | |
| category) | | | | |
| Resistant starch (see listing below under | | | | |
| “Miscellaneous” category) | | | | |
| Inulin (see listing below under “Miscellaneous” | | | | |
| category) | | | | |
| Brown rice water (see listing below under | | | | |
| “Miscellaneous” category) | | | | |
| Phytonutrients | | | | |
| Blueberry (whole fruit, juice, vinegar, extract, powder- | 100 | mg | (25-200 | mg) |
| also 4% freeze-dried form, antho-flavonoids) | | | | |
| Curcumin (turmeric root, turmeric extract) | 50 | mg | (5-100 | mg) |
| Monk fruit (luo han guo, raw whole fruit, extract) | 10 | mg | (1-10 | mg) |
| Nitrates (beetroot, powder, extract) | 100 | mg | (5-100 | mg) |
| (beetroot juice) | 90 | ml | (15-120 | ml) |
| Stevia (rebaudioside A, leaf, extract, powder) | 20 | mg | (1-20 | mg) |
| (liquid drops concentrate) | 15 | mg | (1-15 | mg) |
| Electrolytes | | | | |
| Sodium (chemical or natural form) | 90 | mg | (25-120 | mg) |
| Potassium (chemical or natural form) | 250 | mg | (100-750 | mg) |
| Mineral Co-Factors | | | | |
| Magnesium (citrate, lactate, gluconate, oxide, carbonate, | 25 | mg | (5-40 | mg) |
| hydroxide, chloride, sulfate, lactic acid, phosphate, | | | | |
| tribasic phosphate, natural forms) | | | | |
| Selenium (L-selenomethionine, sodium selenite/selenate, | 20 | mg | (5-30 | mg) |
| natural forms) | | | | |
| Zinc (glycinate, gluconate, oxide, sulfate, natural forms) | 1 | mg | (0.1-1.1 | mg) |
| Dietary Vitamins | | | | |
| Vitamin A (retinyl palmitate, fish oils, natural sources) | 1,000 | IU | (500-1,250 | IU) |
| Vitamin C (calcium ascorbate, ascorbic acid, citrus, rose | 50 | mg | (10-50 | mg) |
| or berry sources) | | | | |
| Vitamin B6 (pyridoxine hydrochloride or yeast sources) | 1.0 | mg | (0.1-1.0 | mg) |
| Vitamin B12 (methylcobalamin, cyanocobalamin, yeast | 1.0 | mg | (0.1-1.0 | mg) |
| sources) | | | | |
| Vitamin B9 (folate, folic acid, folacin, yeast or liver | 100 | mg | (25-100 | mg) |
| sources) | | | | |
| Antioxidants | | | | |
| N-acetyl cysteine (L-cysteine or natural sources: soy | 25 | mg | (10-50 | mg) |
| extract, sesame or flaxseeds, oat bran, wheat germ, | | | | |
| Swiss cheese, peas or whole grain extracts) | | | | |
| Alpha lipoic acid (R+, R−, yeast or liver sources) | 25 | mg | (1-25 | mg) |
| Carotenoids (natural mixed, beta carotene, sea, algal | 2 | mg | (0.1-5 | mg) |
| or plant sources) | | | | |
| Amino Acids | | | | |
| Whey protein | 1,500 | mg | (250-2,500 | mg) |
| L-arginine (chemical or natural form) | 400 | mg | (100-400 | mg) |
| L-carnitine (fumarate or natural form) | 30 | mg | (5-50 | mg) |
| L-citrulline (free or malate) | 250 | mg | (50-500 | mg) |
| L-glutamine (free, alanyl-L-glutamine, trans alanyl- | 250 | mg | (10-400 | mg) |
| L-glutamine) | | | | |
| Nucleotides | | | | |
| Complexes (chemical forms or natural sources: e.g. yeast) | 55 | mg | (0.5-55 | mg) |
| (adenosine monophosphate) | 10 | mg | (0.1-10 | mg) |
| (guanosine monophosphate) | 16 | mg | (0.1-16 | mg) |
| (cytidine monophosphate) | 15 | mg | (0.1-15 | mg) |
| (uridine monophosphate) | 9 | mg | (0.1-9 | mg) |
| (thymidine monophosphate) | 5 | mg | (0.1-5 | mg) |
| D-ribose (natural source, powder, capsules, tablets) | 1,500 | mg | (100-2,500 | mg) |
| Miscellaneous | | | | |
| Omega-3 fatty acids (oils, seeds, yeast, extract) | 250 | mg | (100-500 | mg) |
| Resistant starch | 7 | gms | (1-8 | gms) |
| Inulin (chicory, fructans, oligofructose) | 9 | gms | (1-9 | gms) |
| Brown rice water | 120 | gms | (10-120 | gms) |

For youth utilizing this formulation, it is intended that the commercial product platforms contain at least one constituent from each of at least four ingredient categories.

### Method f the Invention

To provide further enhancement to the unique nature of this invention, the ingredients and combinations included in the formulations are based upon nutritional and antioxidant science. The recommended dosing schedules take into account the pharmacokinetics of the components as related to the desirable clinical effects resulting from a preoperative clear, carbohydrate loading beverage. This commercial application is intended to provide a significant adjunctive modality in achieving the critical goals of the ERAS protocol. These potentially include: 1) metabolic support by reducing insulin resistance and muscle catabolism; 2) diminish fasting-related hunger, thirst, dehydration and stress; 3) improve immune function with lower wound infection rates; 4) decrease oxidative stress and inflammation; 5) reduce postoperative nausea, vomiting and other complications; 6) lessen preoperative morning headache in heavy coffee drinkers; 7) decrease length of stay and hospital costs; and 8) enhance patient satisfaction, functional recovery and quality of life.

To accommodate these pharmacologic and clinical aims, the product dosage is administered in a uniform, standardized and readily tolerable manner. The serving size and combinations within each ingredient category are appropriately based on age. The primary dose listed for each component is intended to represent the usual consumption level but varying circumstances may indicate that utilization of a different dose within the target ranges is preferred. Consumption may also entail none of the ingredients from a particular category, all the ingredients in a category or combinations and mixtures thereof. The Standard Formulation serving size is intended to be a twelve-ounce clear liquid portion. The Youth Formulation serving size is intended to be a four-ounce clear liquid portion. The recommended preoperative regimen suggests two servings around 6 pm (dinner time) the night before surgery and one serving in the morning two to six hours before the procedure as directed by the anesthesiologist or surgeon. This serving regimen may be altered based on the particular patient circumstances or the recommendation of the involved health care providers.

### Category and Ingredient Rationale

This section will discuss the scientific rationale for the use of several components of this invention in the major ingredient categories. The preferred dosage, dose ranges and dosing schedule of consumption has been described previously. Carbohydrates:

Cane sugar, in organic and/or natural form, is less processed and refined compared to white table sugar (sucrose). Therefore, it retains the natural nutrients present in cane juice and avoids the environmental and potentially mutagenic issues related to conventional sugar. For example, natural cane sugar contains beneficial vitamins, minerals and amino acids. In addition, this compound contains higher levels of antioxidants that exhibit a broad spectrum of positive health effects.

Coconut water is a clear liquid containing electrolytes, minerals and phytonutrients that provide human health benefits compared to plain water without interfering in normal hemostatic mechanisms. Of interest in relation to the specific use of this invention, coconut water causes significantly less nausea or fullness compared to some sports beverages that are commonly used for carbohydrate loading in ERAS protocols. The components of coconut water have demonstrated cardio-metabolic benefits in experimental and human studies. The polyphenolic composition of coconut has also demonstrated broad antioxidant qualities in vivo as well as by in vitro experimental models. In addition, there are intriguing in vitro studies showing anti-microbial, anti-viral and anti-tumor effects of coconut water preparations. Similar models have demonstrated cellular and organ preservation qualities, and again particularly relevant to the use of this invention, benefits in glucose metabolism and kidney protection.
D-ribose (see listing below under “Nucleotides” category)
Resistant starch (see listing below under “Miscellaneous” category)
Brown rice water (see listing below under “Miscellaneous” category)
Inulin (see listing below under “Miscellaneous” category)

### Phytonutrients

Species of blueberry fruits (genus Vaccinium) contain polyphenols and anthocyanidin molecules which are powerful antioxidant compounds. These phytochemicals are readily soluble and bioavailable in many forms. Their bioactivity in terms of free radical scavenging and anti-inflammatory effect have been well documented. The interplay between inflammation and obesity may provide another intervention target for blueberry fruits. In addition, this phytonutrient has shown potential efficacy as an anti-cancer radiosensitizer. Of particular importance has been the emergence of blueberry polyphenols and anthocyanins as neuroprotective agents. Experimental and clinical studies have documented memory enhancement and neurocognitive improvement. Recent animal model investigations have elucidated likely mechanisms that are responsible for the observed neuroplasticity.

Curcumin is a phytonutrient abundantly present in the spice, turmeric, and demonstrates a wide spectrum of therapeutic properties. Its potential application to lung protection from hazardous exposures has been described. Immune enhancement is another potential benefit. This polyphenolic agent decreases muscular pain and reduces inflammation and post-exercise muscle damage in human subjects. It improves in vivo neural functioning and protects against oxidative stress and brain ischemia-reperfusion injury. Curcumin has also shown benefit against oxidative damage, and in combination with resveratrol, demonstrates unique radioprotective qualities on normal tissues. In this invention, the characteristics of this nutrient may be relevant for improving patient preoperative health status.

Monk fruit (Siraitia grosvenorii) and stevia (Stevia rebaudiana) are natural non-nutritive sweeteners that are alternatives to sugars or artificial sweeteners. They are generally regarded as safe, contain no calories, carbohydrates or fats and do not have the negative side effects common to artificial compounds. The sweetness from these ingredients is derived from specific glycosides, mogrosides in monk fruit and Rebaudioside A in stevia. These fruits are about 200 times sweeter than sucrose, appear to have acceptable taste profiles and are preferred on product labels as “natural”. Mogrosides have demonstrated antioxidant properties and are related to novel metabolic pathways. In fact, anti-cancer activity has been shown in experimental models. Both these natural sweeteners have low glycemic indices and do not cause glucose level spikes or different insulin responses when compared to sucrose.

Dietary nitrates have engendered much interest as a protective nutrient. Potential health benefits in a variety of settings have been emerging scientifically especially related to high nitrate vegetable intake. Beetroot (Beta Vulgaris L.) juice has been identified as having among the highest concentrations of nitrates, and yet it is also known to contain many other bioactive constituents including antioxidants and polyphenols. In fact, oxidative stress reduction appears to be a central component of the organ protective effects. While public consumption and experimental studies have utilized sodium nitrate, beetroot and other sources, it is recognized that nitrates from natural vegetables are generally considered safe. Relative to this invention and the importance of pre-habilitation in ERAS protocols, it is the exercise benefits of nitrates that has drawn attention. Beetroot juice and sodium nitrate enhance muscle efficiency, reduce oxygen consumption and improve exercise tolerance even for extended time periods. These observations have been confirmed in several direct human trials. The underlying mechanisms are being elucidated in experimental settings.

### Electrolytes

These vital salts dissolve in polar solvents such as water or other bodily fluids to conduct electrical impulses throughout the body. They are essential for biochemical reactions, organ function and energy production. Two ubiquitous electrolytes are sodium and potassium which help control fluid balance inside cells and in the extracellular fluid compartments of the body. Potassium is the most common intracellular electrolyte while sodium is the main one found outside the cells. Precise balance between these two are critical for muscle and nervous system function via cell membrane transport. They are also important in maintaining the proper pH of the blood. Relative depletion from not drinking for prolonged time periods may cause symptomatic deficiency and electrolyte imbalance may be caused by several chronic diseases. Including sodium and potassium in the formulation for this invention and as part of an ERAS protocol provides a modicum of protection against the mild dehydration that some patients would otherwise experience preoperatively.

### Mineral Co-Factors

Magnesium holds a prominent position among essential elements that function as required co-factors for critical enzymatic reactions, biochemical function and metabolic pathways. Its supplementation is generally safe and is an effective therapeutic intervention in many clinical situations. It may also have a role as an electrolyte. One of the prime targets of ERAS protocols is control of postoperative pain and this mineral has been shown to be beneficial in this regard by extensive analysis of human clinical trials. In addition, magnesium supplementation has recently been reported to provide preventive symptom effects in gynecology and to reduce complications in cardiac surgery.

Selenium is a co-factor of glutathione peroxidase, and Se-glutathione peroxidase also acts as an antioxidant. This mineral also decreases harmful effects of pro-inflammatory cytokines and is radio-protective. Recent experimental studies have shown that selenium may protect against DNA damage and inhibit tumor progression. Therefore, selenium supplementation at adequate but safe doses is essential for promoting antioxidant enzyme systems and reducing potential oxidative stress.

As has been emphasized in this section, trace elements play an important role in human health. Zinc is an essential micronutrient in this regard and functions as a biological catalyst and regulator with critical effects in oxidative stress, immune function, cellular integrity and the aging process. In fact, since zinc levels decline with age, there are broad international efforts to ascertain appropriate supplementation guidelines. In this population, zinc is vital to maintenance of DNA repair and genomic stability. Zinc also provides a protective antioxidant function against peripheral neuropathy in diabetic animal models. Several chronic diseases related to oxidative stress and inflammation are associated with inadequate zinc levels.

### Dietary Vitamins

Vitamin C prevents lipid oxidation and potentiates nitric oxide activity in normalizing vascular function in patients with cardiovascular disease associated with high blood pressure, high cholesterol levels and in smokers. Vitamin C is necessary to protect cellular components in aqueous environments and plays an important role in maintaining cellular levels of vitamin E by recycling the vitamin E radical (oxidized) to the reduced (antioxidant) form. Recent studies have elucidated the metabolic and genetic cellular mechanisms in hematopoietic stem cells. Mutations in these cells can lead to blood cancers and vitamin C appears to restore normal cellular regulation and may reduce cancer risk and suppress cancer progression. Vitamin C as calcium ascorbate is beneficial because it is the most effective non-acidic form available for human use and, therefore, is less likely to cause stomach upset and diarrhea. Vitamin C (in combination with other antioxidant vitamins) produces a synergistic inhibition on LDL-cholesterol oxidation.

The most important functions of vitamin B6, pyridoxine, are related to the health of the nervous and immune systems. Specifically, this vitamin is involved in the biosynthesis of neurotransmitters thereby impacting cognitive development. Deficiency is associated with several clinical conditions resulting in low plasma concentrations. While adequate levels have not definitively shown effect in cancer or cardiovascular disease, there may be some benefit in neurocognitive domains.

Vitamin B12 is a water-soluble vitamin that is necessary for neurological function, DNA synthesis and red cell formation as well as acting as an important enzymatic co-factor. As with other B vitamins, deficiency syndromes can show a wide spectrum of symptoms. Older individuals are especially affected. Because of its cognitive effects, vitamin B12 continues to be assessed in dementias.

Folate and its folic acid form are critical in the synthesis of nucleic acids and amino acid metabolism through its function as a coenzyme. Deficiency is generally related to inadequate food or supplement intake and is represented by a wide spectrum of symptoms. Folate deficits are associated with bowel cancer risk and supplementation may reduce this. Similar to other B vitamins, folate's effect in heart disease is inconsistent but may be protective against vascular-related ischemic stroke. In addition, several studies have shown benefit in individuals with depression. These findings may have particular relevance in the preoperative preparation of surgical patients in ERAS protocols.

### Antioxidants

Glutathione, a sulfhydryl compound, is the body's primary endogenously formed antioxidant. It can catabolize anions and hydrogen peroxide and is a potent intracellular protective agent against excess free radical damage. In addition, reduced glutathione actively protects both normal and cancer cells against radiation damage. Specifically, sulfhydryl compounds protect against radiation injury to the bone marrow and gastrointestinal system as well as against cellular mutagenesis and can induce radiation resistance. However, when glutathione is consumed orally by humans, its plasma levels do not significantly increase suggesting that this tripeptide is completely hydrolyzed in the intestinal tract during digestion.

Nevertheless, two other agents, N-acetylcysteine and alpha lipoic acid, have been shown to actively increase intracellular glutathione levels by different non-competing mechanisms. They are well tolerated in humans, are rapidly absorbed without toxicity and have demonstrated radio-protective value. This further confirms their antioxidant value since it is estimated that the majority of the cellular damage from radiation is due to excess free radicals. These agents also restore glutathione in endothelial cells and cardiac muscle that suffered oxidative damage with resultant beneficial effects. Alpha-lipoic acid also has some unique functions in relation to metabolic disorders. It improves glucose utilization in peripheral tissue by stimulating glucose transport and uptake, thereby diminishing insulin resistance and decreases related complications by reducing protein glycation and oxidative stress.

Beta-carotene, the precursor of vitamin A is an essential antioxidant that decreases free radical-induced oxidative stress. These two agents must be included individually because they each possess specific biological functions not provided by the other. Vitamin A is critical for induction of beneficial cellular differentiation while beta-carotene increases the expression of the connexin gene, which encodes for a gap junction protein that is necessary in maintaining normal cellular phenotypes. It is also more effective in quenching oxygen-derived free radicals. Both these antioxidants protect cellular components in lipid environments. The natural “trans” isoform of vitamin A is biologically active and only the natural form of beta-carotene can effectively decrease radiation-induced cellular abnormalities.

### Amino Acids

This category of agents is critical because of their biological characteristics and could be considered of value in a preoperative beverage as part of an ERAS protocol. Chains of amino acids make up peptides which are considered bioactive compounds. These agents, when part of preoperative oral feeding regimens, engender beneficial effects in postoperative metabolism. Long continuous unbranched chains of peptides are known as polypeptides. When the chain contains approximately 50 amino acids, it is designated a protein. A bioactive protein is a “straight” polypeptide chain that is folded and optically arranged into a three-dimensional biologically functional configuration. Essential amino acids are not made by the body and must be consumed and those that contain branched chains demonstrate some important qualities.

These branched chain varieties appear to show benefit in exercise and muscle metabolism. Their mechanisms of organ protection have been elucidated. Positive effects in cardiometabolic health have also recently been demonstrated. An effective method for delivering essential (including branched chain) amino acids is through whey protein consumption as utilized in this invention. Whey may be supplemented in several forms, including concentrates, isolates or hydrolysates depending on alteration of protein and fat content, bioactive component levels, pre-digestion and absorption. Whey provides a number of benefits in relation to muscle function and protect ion. It also has been shown to improve cardiovascular status in high risk populations.

L-arginine is the direct substrate for the family of nitric oxide synthases and is involved in metabolic processes of immune cells and lymphocyte function. As expected, this amino acid also increases nitric oxide production with the resultant promotion of vascular dilation. It is further anticipated that muscle strength and recovery will be improved and provide ergogenic benefit. These expectations have been demonstrated in human studies where exertion-related oxygen consumption was reduced and exercise tolerance enhanced. In addition, oxidative damage can be decreased, immune function enhanced and organ function improved in experimental models. Of note, L-arginine and L-citrulline may be synergistic in these effects, including in untrained yet fairly healthy individuals. L-citrulline is also included in this invention and is described in a subsequent section. The potential of positive effects from this combination of amino acids exists for mitochondrial disease and psychological depressive states.

Only the natural “L” form of (acetyl-, proprionyl-) L-carnitine is biologically active in humans. This agent is included in the invention because of its role in performance, inflammation, pain, oxidative stress and neuroprotection. L-carnitine also shows beneficial effects in lipid metabolism and potentially in diabetes and obesity. It is safe and well-tolerated for extended time periods and the crosses the blood brain barrier. Various forms have demonstrated positive clinical benefits in vascular and neurodegenerative conditions. It can also protect against radiation-induced sensory organ and brain damage. In addition, L-carnitine and alpha lipoic acid synergistically enhance mitochondrial function. Finally, in relevance to surgical patients in ERAS protocols, this amino acid may reduce the severity of constipation.

As implied above, L-citrulline is closely related to the metabolism of L-arginine. Since L-citrulline is not subject to hepatic degradation, it is metabolized into arginine and can enhance those levels effectively and play a role in protein synthesis and nitric oxide metabolism. The effect of increased nitric oxide on human performance has been extensively evaluated. Performance during exertion and recovery are positively influenced by L-citrulline in clinical trials of healthy males and athletes. This has direct implications to preoperative functional capacity for patients in ERAS protocols. In addition, the vascular effects of this amino acid have therapeutic effects in heart disease and cold exposure stress. The benefits also seem to extend to the elderly in both clinical and experimental situations. Finally, L-citrulline seems to exhibit beneficial influences in ischemic conditions and liver damage.

Glutamine is the most abundant amino acid in the human body and has been considered an ergogenic compound with benefits also in regard to the immune system and intestinal health. It has several biologic functions including nitrogen transport and acid/base metabolism as well as being a precursor for nucleotides and glutathione, the body's most ubiquitous antioxidant. It also is a substrate for L-citrulline, enhancing the production of that important amino acid as noted in the previous section. In these roles, glutamine has demonstrated protective effects against cardiac tissue damage. Of particular importance to this invention and preoperative carbohydrate loading, glutamine appears to be able to diminish the spikes in blood glucose and insulin levels in response to dietary carbohydrate ingestion. This compound also functions as an energy substrate for immune cells and may improve their function in critical clinical situations.

### Nucleotides

Preservation of immune status is a desirable metabolic outcome after major surgery. Nucleotides are considered strong immune enhancer candidates and are bioactive compounds that contribute to critical human physiologic processes. They are the building units of nucleic acids and involved in energy transfer, cellular communication and co-enzyme catalysts. They play an important role in organ protection. Recent innovations in imaging techniques have defined their potential function in neuronal and cardiovascular health. In addition, they may interact with the natural product, curcumin (see “Phytonutrients” section above), to provide anticancer effects.

The pentose sugar, D-ribose is a critical component of nucleotides. D-ribose has shown metabolic support of cardiac energy and improves functional patient outcomes and quality of life. In combination with other agents, it also provides cellular protection of the heart in animal models. These findings parallel the beneficial experience of using D-ribose to enhance cardiac function in heart surgery and cardiomyopathy. In further relation to use in the present invention, improvement after exertion with supplementation of this nucleotide subunit may generally be anticipated and its role in immune modulation is also evident. D-ribose demonstrates beneficial reduction of oxidative stress in animal and human studies. In addition, in vivo models have shown neuroprotection and enhanced wound healing. Finally, this supplement has been in generally safe public usage for an extended period of time and recent experimental studies have supported this impression.

### Miscellaneous Nutrients

Caffeine has interesting potential roles in the preoperative beverage as a part of ERAS protocols. Among the concerns for patients is the anxiety related to surgery which may result in diminished sleep and mild dehydration. However, when patients who are heavy coffee drinkers are restricted from their usual consumption, they often suffer severe preoperative headaches which may complicate the anesthesia preparation. A modest caffeine dose as part of the preoperative beverage would be expected to abrogate this significant unpleasant symptom. In addition, there are other potential positive effects from caffeine. If individuals are at all sleep-deprived, caffeine may influence alertness, recovery sleep and mental fatigue. In this setting, it may also improve functional performance and decrease pain perception.

Diet and supplementation are excellent sources of omega-3 fatty acids. These substances have demonstrated positive health effects in animal models and in human clinical conditions. They have also shown benefit in vivo against ultraviolet radiation-induced oxidative stress. The omega fatty acid constituents decrease inflammation biomarkers, muscle soreness and blood pressure after exercise. Recent studies demonstrate that this potent anti-inflammatory effect on the immune and nervous systems is mediated through endocannabinoids. Furthermore, In vivo and human studies show neurological effects from the fatty acids such as enhanced brain development and improved spatial memory. In specific regard to surgical patients in ERAS protocols, preoperative omega-3 fatty acid supplementation may reduce the risk of cardiac rhythm disturbances. Most recently, the differential effects of the primary omega-3 fatty acids in fish oils, docosahexaenoic acid and eicosapentaenoic acid, have been delineated in human trials post-exercise and in vascular risk prevention. Combining these agents with whey protein supplementation (see “Amino Acids” section above) also provides a benefit following exertion.

Additional carbohydrate sources have also been included in this invention and have been listed earlier in the “Standard” and “Youth” Formulations. Because of their optional utilization, they are listed in the “Miscellaneous” category of the formulations. Therefore, they are further described by narrative here in this “Miscellaneous Nutrients” part of the “Category and Ingredient Rationale” section of the invention.

Resistant starches are naturally-occurring complex carbohydrate sources that generally are not digested or absorbed in the small intestine and are only acted upon in the colon. In this site, bacterial fermentation of resistant starch forms short-chain fatty acids such as butyrate which has shown protective effects. “Prebiotic” effects of this ingredient can be seen in the modification of the microbiota of the large intestine including reduction of pathogenic gut flora and enhancement of healthful bacterial strains. Considerable positive health benefits include metabolic, oncogenic and cardiovascular at dose levels that may only cause mild bloating or intestinal gassiness. In addition, this form of slowly absorbed carbohydrate has a favorable influence on decreasing human glycemic responses to ingestion.

Brown rice-steeped water parallels the nutritional effects of brown rice, a whole grain carbohydrate that exhibits a variety of health benefits. It is well known that its intake is nutritious and associated with higher levels of fiber and polyunsaturated fatty acids. The grain contains great antioxidant capacity due to the presence of anthocyanins, flavonoids and phenolic compounds. The positive effects on cholesterol levels and cardiovascular risk have also been demonstrated in a wide age range of individuals. Benefit is likewise noted in metabolic syndrome and diabetes. Finally, this carbohydrate source may have chemo-preventive and protective qualities. Its high selenium content may contribute to these findings.

Inulin is a natural complex carbohydrate of the dietary fiber fructan variety with the character of a resistant starch as defined previously in this section. It is a heterogenous combination of fructose polymers and can be extracted from many plants, most commonly, chicory. It has been described as an original “functional food” since it can maintain health and may reduce some disease risk. Because inulin is not digested or absorbed in the upper gastrointestinal tract, it has little effect on serum glucose levels and has a caloric value less than half of that of other carbohydrates. This form of dietary fiber has shown no toxic effects and has long been considered a GRAS (generally accepted as safe) food in virtually all countries. Like other resistant starches, inulin is fermented in the colon to acetate, butyrate and lactate and can stimulate the growth of favorable bacteria such as bifidobacteria while reducing potentially harmful species and toxic metabolites. Scientific reviews have outlined many of the benefits of inulin especially with regard to digestive health, lipid metabolism, mineral absorption, glucose homeostasis and oncogenesis. Further studies have again emphasized positive effects in “prebiotic” function and cardiovascular risk.

### Impact of the Invention

Oral consumption of micronutrients and phytochemicals is exposed to variations in pharmacodynamics, pharmacokinetics and bioavailability. Therefore, delivery methods, dosages, dose ranges and schedules must have broad applicability. In this invention, the primary doses and specific categories of ingredients were targeted to the unique situation of a preoperative carbohydrate loading beverage as it relates to an overall Enhanced Recovery After Surgery (ERAS) strategy. The dosage and dose ranges were further based on age groups. The dose schedules were also tailored to the requirements of the different pre-surgical timing scenarios. The ingredient dose ranges were particularly designed to be adequately broad and at sufficient levels to provide the desired effects in humans of both genders and of wide weight cohorts. The included categories to which the components of the present formulations belong are known to fulfill ERAS goals in relation to metabolic effect, preoperative stressors, immune support, oxidative damage and inflammation, perioperative symptomatology and functional outcomes as well as length of hospital stay and health care cost. While this invention describes a delivery platform of a liquid beverage, it is intended to also relate to any form of administration of these formulations in humans including but not limited to capsules, tablets, bars, powders, gummies, lozenges/troches, dissolvable disks, chewables, inhalational forms, injectables, patches, ointments, gels, aerosols, and sticks as well as any transcutaneous, intranasal, intra-cavitary, liposomal, nanotechnology or other delivery system.

Numerous modifications and variations of the present invention are possible in light of the above teachings. It is therefore to be understood that within the scope of the attendant claims.

What is claimed is:

1. A formulation comprising at least one phytonutrient, at least one mineral, at least one dietary vitamin, at least one antioxidant, at least one carbohydrate, at least one electrolyte, at least one amino acid, and at least one other nutrient, and mixtures and combinations thereof.

2. The formulation of claim 1 wherein said carbohydrate is selected form a group comprising coconut water, cane sugar, D-ribose, simple sugars, resistance starch, brown rice water, inulin, complex carbohydrates, and combinations thereof.

3. The formulation of claim 1 wherein said electrolyte is selected from a group comprising sodium, potassium, chloride, bicarbonate, magnesium, calcium, phosphate, and combinations thereof.

4. The formulation of claim 1 wherein said amino acid is selected from a group comprising glutamine, amino acid complex (whey protein), arginine, carnitine, citrulline, alanine, valine, leucine, isoleucine, phenylalanine, tryptophan, methionine, proline, glycine, serine, threonine, cysteine, tyrosine, asparagine, lysine, histidine, and combinations thereof.

5. The formulation of claim 1 wherein said phytonutrient is selected from a group comprising of Bergamot, Blueberry, Cannabinoids, Chokeberry, Cinnamon, Curcumin, Flaxseed, Garlic, Gensing, Ginger, Gingko biloba, Grape juice, Monk Fruit, Moringa, nitrates, Resveratrol, Spearmint, Spirulina, Stevia, Tea, and mixtures and combinations thereof.

6. The formulation of claim 1 wherein said mineral is selected from a group comprising Calcium, Choline, Chromium, Iodine, Magnesium, Selenium, Zinc, and mixtures and combinations thereof.

7. The formulation of claim 1 wherein said dietary vitamin is selected from a group comprising Vitamin A, Vitamin B1, Vitamin B2, Vitamin B3, Vitamin B5, Vitamin B6, Vitamin B7, Vitamin B9, Vitamin B12, Vitamin C, Vitamin D, and mixtures and combinations thereof.

8. The formulation of claim 1 wherein said antioxidant is selected from a group comprising Vitamin E, Coenzyme Q10, Natural mixed carotenoids, Alpha-lipoic acid, L-cysteine, N-acetyl cysteine, and mixtures and combinations thereof.

9. The formulation of claim 1 wherein said other nutrients is selected from group comprising of Lutein, Lycopene, caffeine, omega 3 fatty acids, and mixtures and combinations thereof.

10. The formulation of claim 1 comprises nucleotides, said nucleotides are selected from a group comprising adenosine monophosphate, guanosine monophosphate, cytidine monophosphate, uridine monophosphate, thymidine monophosphate, and combinations thereof.

11. The formulation of claim 1 is for humans age 14 and older.

12. The formulation of claim 1 further comprises caffeine.

13. A formulation comprising:
Cane sugar in a range from about 1 gm to about 50 gms
Coconut water in a range from about 2 gms to about 1,440 gms
D-ribose in a range from about 100 mg to about 15,000 gms
Resistance starch from about 1 gm to about 45 gms
Brown rice water from about 10 gms to about 1440 gms
Inulin from about 1 gm to about 45 gms
Blueberry in a range from about 25 mg to about 1600 mg
Monk fruit in a range from about 1 mg to 5,000mg
Nitrates in a range from about 5 mg to 4,000 mg
Stevia in a range from about 1 mg to 2,000 mg
Sodium in a range from about 25 mg to 2,000 mg
Potassium in a range from about 100 mg to 5,000 mg
Whey protein in a range from about 250 mg to 10,000 mg
L-arginine in a range from about 100 mg to about 6,000 mg
Omega-3 fatty acid in a range from about 100 mg 6,000 mg
Caffeine in a range from about 0 mg to 750 mg
Curcumin in a range from about 5 mg to about 1,800 mg
Magnesium in a range from about 5 mg to about 500 mg
Natural mixed carotenoids in a range from about 0.1 mg to about 60 mg
Selenium in a range from about 5 mcg to about 100 mcg
Zinc in a range from about 0.1 mg to about 15 mg
Alpha-lipoic acid in a range from about 1 mg to about 150 mg
L-carnitine in a range from about 5 mg to about 1,000 mg
L-citrulline in a range from about 50 mg to 7,000 mg
L-glutamine in a range from about 10 mg to about 10,000 mg
N-acetyl cysteine in a range from about 10 mg to 500 mg
Vitamin A from a range of about 500 IU to about 5,000 IU
B6 in a range from about 0.1 mg to about 10 mg
B9 in a range from about 25 mcg to about 1,000 mcg
B12 in a range from about 0.1 mcg to about 10 mcg
Vitamin C in a range from about 10 mg to about 2,000 mg.

13. The formulation of claim 13 further comprises Nucleotides complexes from about 0.1 mg to about 15,000 mg.

14. The formulation of claim 13 further comprising caffeine.

15. A method of manufacturing a formulation, said method comprises admixing at least one phytonutrient, at least one mineral, at least one dietary vitamin, at least one antioxidant, at least one carbohydrate, at least one electrolyte, at least one amino acid, and at least one other nutrient.

16. The method of claim 15, further comprising admixing caffeine.

17. The method of claim 15 further comprising admixing Nucleotides complexes.

18. The method of claim 15 provides for a formulation for humans ages 4 and up.
